(19) United States
(12) Patent Application Publication
(10) Pub. No.: US 2022/0340506 A1
(43) Pub. Date: Oct. 27, 2022

(54) METHOD FOR PRODUCING 1,3-BUTADIENE

(71) Applicants: JSR Corporation, Minato-ku (JP); ENEOS Corporation, Chiyoda-ku (JP)

(72) Inventors: Junjie WANG, Minato-ku (JP); Yuichiro SASAKI, Minato-ku (JP); Takashi MORI, Minato-ku (JP)

(73) Assignees: JSR Corporation, Minato-ku (JP); ENEOS Corporation, Chiyoda-ku (JP)

(21) Appl. No.: 17/639,529

(22) PCT Filed: Aug. 26, 2020
(86) PCT No.: PCT/JP2020/032165

§ 371 Date: Mar. 1, 2022

(30) Foreign Application Priority Data
Sep. 2, 2019 (JP) 2019-159591

(51) Int. Cl. C07C 11/167; B01J 23/887; C07C 5/48

(52) U.S. Cl. CPC C07C 11/167; B01J 23/887; C07C 5/48; C07C 2529/26; C07C 2529/78

(57) ABSTRACT

The present invention provides a method for producing 1,3-butadiene that is capable of suppressing generation of reaction by-products. The method includes: a step (A) of to obtain a produced gas containing 1,3-butadiene; a step (B) of cooling the produced gas; and a step (C) of separating the produced gas cooled in the step (B) into molecular oxygen and inert gases, and other gases containing 1,3-butadiene, by selective absorption into an absorption solvent. In the method, in the step (A), the raw material gas and a molecular oxygen-containing gas are supplied to a fixed-bed reactor with a composite oxide catalyst containing molybdenum and bismuth; the molar ratio of molecular oxygen to n-butene in the gases is 1.0 to 2.0; and the molar ratio of water vapor to n-butene in the gases supplied to the fixed-bed reactor is not more than 1.2.

### TECHNICAL FIELD

The present invention relates to a method for producing 1,3-butadiene, and in particular, relates to a method for producing 1,3-butadiene using an oxidative dehydrogenation reaction.

### BACKGROUND ART

Conventionally, as a method for producing 1,3-butadiene (hereinafter also simply referred to as “butadiene”), there has been adopted a method in which components other than butadiene are separated by distillation from a fraction that is obtained by cracking of naphtha and contains molecules with four carbon atoms (hereinafter also referred to as “C4 fraction”).

Demand for butadiene as a raw material for a synthetic rubber or the like has increased, but the amount of C4 fraction supplied has decreased due to a circumstance in which a method for producing ethylene has been changed from a method based on cracking of naphtha to a method based on pyrolysis of ethane. Therefore, production of butadiene in which the C4 fraction is not adopted as a raw material is required.

Regarding methods for producing butadiene, attention has been paid to a method in which butadiene is isolated and obtained from a produced gas that has been obtained by oxidative dehydrogenation of n-butene (for example, see Patent Literatures 1 to 4). This production method includes an oxidative dehydrogenation step of performing an oxidative dehydrogenation reaction of a raw material gas containing n-butene and a molecular oxygen-containing gas containing molecular oxygen (for example, air), a cooling step of cooling a produced gas obtained in the previous step, and a produced gas separating step of isolating butadiene from the produced gas cooled in the previous step.

In such a method for producing butadiene, the composition of the raw material gas which is subjected to the oxidative dehydrogenation step is generally adjusted by an inert gas and water vapor in such a manner that the concentration of the raw material gas is out of an explosive range.

### CITATION LIST

### Patent Literature

Patent Literature 1: Japanese Patent Application Laid-Open No. 2016-135470
Patent Literature 2: Japanese Translation of PCT Patent Application Publication No. 2016-500372
Patent Literature 3: Japanese Translation of PCT Patent Application Publication No. 2017-502988
Patent Literature 4: Japanese Translation of PCT Patent Application Publication No. 2016-524011

### SUMMARY OF INVENTION

### Technical Problem

However, in the aforementioned method for producing butadiene, reaction by-products such as carbonyl compounds including acetaldehyde or methyl vinyl ketone, and organic acids including carboxylic acid are generated. The generation of such reaction by-products not only causes a reduction in yield of butadiene to be obtained, but also causes a reduction in purification efficiency, for example, by attachment of the reaction by-products to a desolvation tower (reboiler) used in a desolvation process after the produced gas separating step, or causes an increase in load on a wastewater treatment of a cooling medium containing the reaction by-products in the cooling step. Therefore, a method for producing butadiene with suppressed generation of the reaction by-products has been desired.

The present invention has been made in view of the foregoing circumstances and has as its object the provision of a method for producing 1,3-butadiene that is capable of suppressing generation of reaction by-products such as carbonyl compounds and organic acids.

### Solution to Problem

A method for producing 1,3-butadiene of the present invention includes:

a step (A) of performing an oxidative dehydrogenation reaction, with oxygen in the presence of a metal oxide catalyst, of a raw material gas, which contains n-butene, to obtain a produced gas containing 1,3-butadiene;

a step (B) of cooling the produced gas obtained in the step (A); and

a step (C) of separating the produced gas cooled in the step (B) into molecular oxygen and inert gases, and other gases containing 1,3-butadiene, by selective absorption into an absorption solvent, wherein:

in the step (A), the raw material gas and a molecular oxygen-containing gas, as a minimum, are supplied to a fixed-bed reactor of which the inside includes a composite oxide catalyst containing molybdenum and bismuth;

the molar ratio of molecular oxygen to n-butene (molecular oxygen/n-butene) in the gases supplied to the fixed-bed reactor is 1.0 to 2.0; and the molar ratio of water vapor (water) to n-butene (water/n-butene) in the gases supplied to the fixed-bed reactor is not more than 1.2.

In the method for producing 1,3-butadiene of the present invention, it is preferable that in the produced gas obtained in the step (A), the yield of a carbonyl compound is not more than 1.34 mol %, and the yield of a heterocyclic compound is not more than 3.01 mol %.

In the method for producing 1,3-butadiene of the present invention, it is preferable that in the step (B), the produced gas comes into contact with a cooling medium so that the produced gas is cooled, and that the yield of organic acids in the cooling medium that has been in contact with the produced gas is not more than 2 mol %.

In the method for producing 1,3-butadiene of the present invention, it is preferable that in the step (A), the molar ratio of water vapor to n-butene in the gases supplied to the fixed-bed reactor is not more than 0.6.

### Advantageous Effects of Invention

According to the method for producing 1,3-butadiene of the present invention, the molar ratio of water vapor to n-butene in the gases supplied to the fixed-bed reactor is not more than 1.2, and so the generation of reaction by-products such as carbonyl compounds and organic acids can be suppressed.

### BRIEF DESCRIPTION OF DRAWINGS

FIG. 1 is a flow diagram illustrating an example of a specific procedure for performing a method for producing butadiene of the present invention.

### DESCRIPTION OF EMBODIMENTS

Embodiments of the present invention will be described below.

The method for producing butadiene (1,3-butadiene) of the present invention has steps shown in (1) to (3) below, and is to produce butadiene from a raw material gas containing n-butene by performing the steps (1) to (3) described below.

(1) A step (A) of performing an oxidative dehydrogenation reaction, with oxygen in the presence of a metal oxide catalyst, of a raw material gas, which contains n-butene, to obtain a produced gas containing 1,3-butadiene;

(2) A step (B) of cooling the produced gas obtained in the step (A); and

(3) A step (C) of separating the produced gas cooled in step (B) into molecular oxygen and inert gases, and other gases containing 1,3-butadiene by selective absorption into an absorption solvent.

In the method for producing 1,3-butadiene of the present invention, in the step (A), the raw material gas and a molecular oxygen-containing gas, as a minimum, are supplied to a fixed-bed reactor inside which a composite oxide catalyst containing molybdenum and bismuth is carried. The molar ratio of molecular oxygen to n-butene (molecular oxygen/n-butene) in the gases supplied to the fixed-bed reactor is 1.0 to 2.0, and the molar ratio of water vapor to n-butene in the gases supplied to the fixed-bed reactor is not more than 1.2.

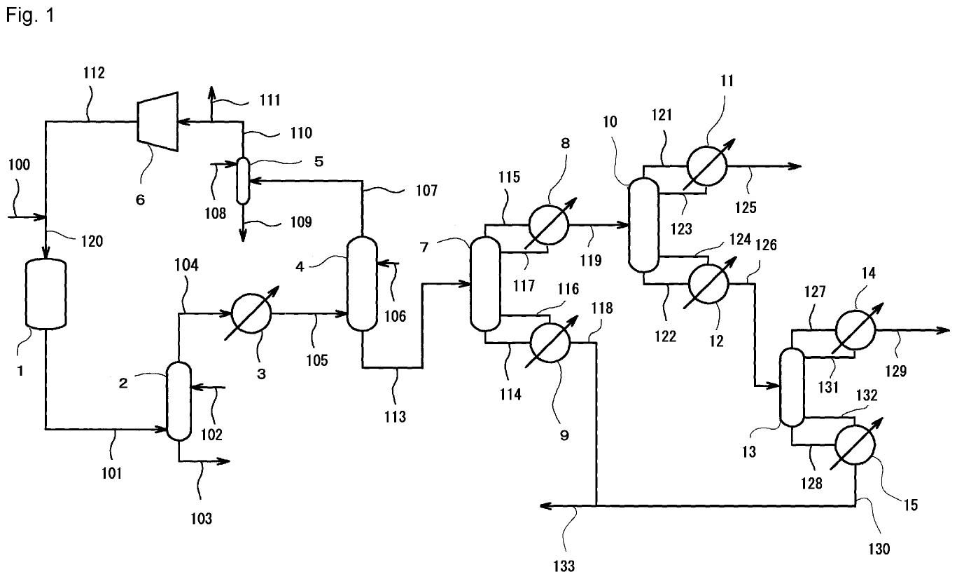

FIG. 1 is a flow diagram illustrating an example of a specific procedure for performing the method for producing butadiene of the present invention.

In the step (C) in the method for producing butadiene exemplified in FIG. 1, the produced gas cooled in the step (B) is separated into molecular oxygen and inert gases, and the other gases containing 1,3-butadiene by selectively absorbing the other gases containing 1,3-butadiene by the absorption solvent.

The method for producing butadiene exemplified in FIG. 1 includes a step (4) below.

(4) A step (D) of separating the absorption solvent, which is obtained in the step (C) and has absorbed the other gases containing 1,3-butadiene, to obtain a 1,3-butadiene liquid containing 1,3-butadiene and the absorption solvent.

Hereinafter, a specific example of the method for producing butadiene of the present invention will be described in detail using FIG. 1.

The method for producing butadiene exemplified in FIG. 1 includes, in addition to the aforementioned steps of (1) to (4), a circulation step of returning the molecular oxygen and the inert gases obtained in the step (C) to the step (A), that is, feeding them as a reflux gas.

Step (A):

In the step (A), the raw material gas and the molecular oxygen-containing gas are subjected to an oxidative dehydrogenation reaction in the presence of a metal oxide catalyst to obtain a produced gas containing 1,3-butadiene. In this step (A), the oxidative dehydrogenation reaction of the raw material gas with the molecular oxygen-containing gas is performed by a fixed-bed reactor 1 as illustrated in FIG. 1. This fixed-bed reactor 1 is a tower-shaped reactor that has a gas inlet provided at an upper portion and a gas outlet provided at a lower portion and includes a catalyst layer (not illustrated) formed by filling the inside of the reactor with the metal oxide catalyst. In this fixed-bed reactor 1, pipings 100 and 112 are connected to the gas inlet via a piping 120, and a piping 101 is connected to the gas outlet.

The step (A) will specifically be described. The raw material gas and the molecular oxygen-containing gas, and inert gases or inert gases and water vapor (hereinafter also collectively referred to as “gas for concentration adjustment”) used as necessary are heated to a temperature between about 200° C. and about 400° C. by a preheater (not illustrated) which is disposed between the fixed-bed reactor 1 and the piping 100. The gasses are then supplied to the fixed-bed reactor 1 through the piping 100 communicating with the piping 120. Together with the raw material gas, the molecular oxygen-containing gas, and the gas for concentration adjustment (hereinafter also collectively referred to as “newly supplied gas”) supplied through the piping 100, the reflux gas from the circulation step is supplied to the fixed-bed reactor 1 through the piping 112 communicating with the piping 120 after being heated by the preheater. That is, a mixed gas including the newly supplied gas and the reflux gas is supplied to the fixed-bed reactor 1 after being heated by the preheater. Herein, the newly supplied gas and the reflux gas may each be supplied directly to the fixed-bed reactor 1 through separate pipings. However, it is preferable that the newly supplied gas and the reflux gas in a mixed state are supplied to the fixed-bed reactor 1 through the common piping 120, as illustrated in FIG. 1. When the common piping 120 is provided, a mixed gas that contains various components and is in a state where the components are uniformly mixed in advance is supplied to the fixed-bed reactor 1. This can prevent a situation where a gas mixed in a nonuniform manner partially forms a detonating gas in the fixed-bed reactor 1.

In the fixed-bed reactor 1 to which the mixed gas has been supplied, butadiene (1,3-butadiene) is produced by an oxidative dehydrogenation reaction of the raw material gas with the molecular oxygen-containing gas, and so a produced gas containing the butadiene is obtained. The obtained produced gas is discharged from a gas outlet of the fixed-bed reactor 1 to the piping 101.

Raw Material Gas:

As the raw material gas, a gaseous substance obtained by gasifying n-butene, which is a monoolefin having 4 carbon atoms, in a vaporizer (not illustrated) is used. This raw material gas is a combustible gas. In the present invention, “n-butene” means linear butenes, and specifically, may include 1-butene, cis-2-butene and trans-2-butene.

In addition, optional impurities may be contained in the raw material gas within a range that does not inhibit the effects of the present invention. Specific examples of the impurities include branched monoolefins such as i-butene, and saturated hydrocarbons such as propane, n-butane and i-butane. In addition, the raw material gas may contain 1,3-butadiene as an impurity, which is the production target. The amount of impurities contained in the raw material gas is usually not more than 60% by volume, and may preferably be not more than 40% by volume, more preferably not more than 25% by volume, particularly preferably not more than 5% by volume, per 100% by volume of the raw material gas. When the amount of impurities is excessively large, the reaction rate tends to be lowered due to the decrease in the concentration of linear butene in the raw material gas, or the amount of by-products tends to increase.

As the raw material gas, for example, a fraction (raffinate 2) containing a linear butene, as a main component, obtained by separating butadiene and i-butene from a C4 fraction (a fraction containing molecules with 4 carbon atoms), which is by-produced by naphtha cracking, or a butene fraction generated by a dehydrogenation reaction or an oxidative dehydrogenation reaction of n-butane may be used. High purity 1-butene, cis-2-butene and trans-2-butene, which are obtained by dimerization of ethylene, and gas mixtures thereof may also be used. In addition, gasses containing a large amount of hydrocarbons having 4 carbon atoms (hereinafter, sometimes abbreviated as “FCC-C4”) may be used as raw material gases as they are, and here the gasses can be obtained through Fluid Catalytic Cracking by cracking a heavy oil fraction, which is obtained when a crude oil is distilled in a petroleum refining plant or the like, using a powdery solid catalyst in a fluidized bed state to convert the heavy oil fraction into hydrocarbons having low boiling points. Gasses obtained by removing impurities such as phosphorus from FCC-C4 may also be used as a raw material gas.

Molecular Oxygen-Containing Gas:

The molecular oxygen-containing gas is usually a gas containing 10 volume % or more of molecular oxygen (O₂). In this molecular oxygen-containing gas, the concentration of molecular oxygen may preferably be not less than 15% by volume, more preferably not less than 20% by volume.

The molecular oxygen-containing gas may include, in addition to molecular oxygen, an optional gas such as molecular nitrogen (N₂), argon (Ar), neon (Ne), helium (He), carbon monoxide (CO), carbon dioxide (CO₂) and water (water vapor). The amount of the optional gas in the molecular oxygen-containing gas is usually not more than 90% by volume, and may be preferably not more than 85% by volume, more preferably not more than 80% by volume when the optional gas is molecular nitrogen, and is usually not more than 10% by volume, and may preferably be not more than 1% by volume when the optional gas is a gas other than molecular nitrogen. When the amount of the optional gas is excessively large, in the reaction system (inside of the fixed-bed reactor 1), molecular oxygen of a required amount may not coexist with the raw material gas. In the step (A), preferred specific examples of the molecular oxygen-containing gas include air.

Inert Gases:

It is preferable that the inert gases constituting the gas for concentration adjustment are supplied to the fixed-bed reactor 1 together with the raw material gas and the molecular oxygen-containing gas.

When inert gases are supplied to the fixed-bed reactor 1, the concentrations (relative concentrations) of the raw material gas and the molecular oxygen can be adjusted in such a manner that the mixed gas does not form a detonating gas in the fixed-bed reactor 1.

Examples of the inert gases used in the method for producing butadiene include molecular nitrogen (N₂), argon (Ar) and carbon dioxide (CO₂). These may be used either singly or in any combination of two or more thereof. Among these, molecular nitrogen is preferred from an economic viewpoint.

Water Vapor:

When water vapor is used as the gas for concentration adjustment, it is preferable that the water vapor is supplied to the fixed-bed reactor 1 together with the raw material gas and the molecular oxygen-containing gas.

By supplying water vapor to the fixed-bed reactor 1, it is possible to adjust the concentrations (relative concentrations) of the raw material gas and the molecular oxygen in the same manner as that in the aforementioned inert gases in such a manner that the mixed gas does not form a detonating gas in the fixed-bed reactor 1.

Mixed Gas:

Since the mixed gas of the raw material gas, the molecular oxygen-containing gas and the gas for concentration adjustment, that is, the entire gas supplied to the fixed-bed reactor 1 contains the combustible raw material gas and the molecular oxygen, the composition thereof is adjusted in such a manner that the concentration of the raw material gas does not fall within an explosive range.

Specifically, the composition of the mixed gas at the gas inlet of the fixed-bed reactor 1 is controlled by monitoring the flow rates with flow meters (not illustrated) installed in the pipings (specifically the piping (not illustrated) communicating with the piping 100, and the piping 112) for supplying respective gases constituting the mixed gas to the fixed-bed reactor 1. For example, depending on the molecular oxygen concentration of the reflux gas supplied to the fixed-bed reactor 1 through the piping 112, the composition of the newly supplied gas to be supplied to the fixed-bed reactor 1 through the piping 100 is controlled.

In this specification, the “explosive range” indicates a range in which the mixed gas has a composition such that it ignites in the presence of some ignition source. Here, it is known that an ignition source that coexists does not ignite a combustible gas when the concentration of the combustible gas is lower than a certain value, and this concentration is referred to as a lower explosion limit. Such a lower explosion limit is the lower limit of the explosive range. It is also known that, when the concentration of the combustible gas is higher than a certain value, an ignition source that coexists does not ignite the combustible gas, and this concentration is referred to as an upper explosion limit. Such an upper explosion limit is the upper limit of the explosive range. These values depend on the concentration of molecular oxygen. In general, the lower the concentration of molecular oxygen is, the more the values approach each other. When the concentration of molecular oxygen becomes a certain value, the values coincide with each other. The concentration of molecular oxygen at this time is referred to as the limit oxygen concentration. Thus, if the concentration of molecular oxygen in the mixed gas is lower than the limit oxygen concentration, the mixed gas does not ignite regardless of the concentration of the raw material gas.

Specifically, in the mixed gas, the concentration of n-butene may preferably be not less than 2% by volume and not more than 30% by volume, more preferably not less than 3% by volume and not more than 25% by volume, particularly preferably not less than 5% by volume and not more than 20% by volume, per 100% by volume of the mixed gas from the viewpoint of productivity of butadiene and suppression of burden on the metal oxide catalyst. If the concentration of n-butene is excessively low, the productivity of butadiene may decrease. On the other hand, when the concentration of the n-butene is excessively large, the burden on the metal oxide catalyst may increase.

In addition, the molar ratio of molecular oxygen to n-butene in the mixed gas is set to 1.0 to 2.0, and may preferably be 1.2 to 1.5. When the molar ratio of molecular oxygen to n-butene deviates from the aforementioned range, adjustment of the concentration of molecular oxygen at the gas outlet of the fixed-bed reactor 1 by adjusting the reaction temperature tends to be difficult. Then, when it becomes difficult to control the concentration of molecular oxygen at the gas outlet of the fixed-bed reactor 1 by the reaction temperature, decomposition of the target product and occurrence of side reactions inside the fixed-bed reactor 1 may not be suppressed.

In the mixed gas, the ratio of the sum of molecular nitrogen and water vapor to the raw material gas may preferably be not less than 400 mol and not more than 2,700 mol, more preferably not less than 500 mol and not more than 2000 mol, per 100 mol of the raw material gas. When the ratio of the sum of molecular nitrogen and water vapor is excessively large, as the value thereof increases, the production efficiency of butadiene tends to decrease due to the decreased concentration of the raw material gas. On the other hand, when the ratio of the sum of molecular nitrogen and water vapor is excessively small, as the value thereof decreases, the concentration of the raw material gas tends to fall within the explosive range, or heat removal of the reaction system, which will be described later, tends to be difficult.

The molar ratio (water/n-butene) of water vapor to n-butene in the mixed gas, that is, the gas supplied to the fixed-bed reactor 1 is set to a value of not more than 1.2, preferably not more than 0.6. When the concentration of water vapor in the mixed gas is excessively large, it is difficult to sufficiently suppress the generation of by-products.

Metal Oxide Catalyst:

As the metal oxide catalyst, a composite oxide catalyst containing molybdenum and bismuth is used. As such a composite oxide catalyst, for example, those containing at least molybdenum (Mo), bismuth (Bi) and iron (Fe) can be used, and specific examples thereof include those containing a composite metal oxide represented by the following composition formula (1).

MOₐBi_{b}Fe_{c}X_{d}YₑZ_{f}O_{g} Composition formula (1):

In the aforementioned composition formula (1), X is at least one selected from the group consisting of Ni and Co. Y is at least one selected from the group consisting of Li, Na, K, Rb, Cs and Tl. Z is at least one selected from the group consisting of Mg, Ca, Ce, Zn, Cr, Sb, As, B, P and W. a, b, c, d, e, f and g each independently show the atomic ratio of each element; when a is 12, b is 0.1 to 8, c is 0.1 to 20, d is 0 to 20, e is 0 to 4, f is 0 to 2, and g is the number of atoms of the oxygen element required to satisfy the atomic valence of each of the aforementioned components.

A composite oxide catalyst containing the composite metal oxide represented by the aforementioned composition formula (1) is highly active and highly selective in a production method of butadiene using an oxidative dehydrogenation reaction, and is further excellent in life stability.

The method for preparing the composite oxide catalyst is not particularly limited, and a known method such as an evaporation and drying method, a spray drying method, or an oxide mixing method using raw materials of respective elements relating to the composite metal oxide constituting the composite oxide catalyst to be prepared can be adopted.

The raw materials of the aforementioned respective elements are not particularly limited, and examples thereof include an oxide, a nitrate salt, a carbonate salt, an ammonium salt, a hydroxide, a carboxylic acid salt, an ammonium carboxylate salt, an ammonium halide salt, a hydrogenated acid and an alkoxide of the component elements.

Furthermore, the composite oxide catalyst may be used while being carried on an inert carrier. Examples of the carrier species include silica, alumina and silicon carbide.

Oxidative Dehydrogenation Reaction:

When an oxidative dehydrogenation reaction is initiated in the step (A), it is preferable that supply of the molecular oxygen-containing gas, inert gases and water vapor to the fixed-bed reactor 1 is first initiated, then the amounts supplied of these are adjusted so that the concentration of molecular oxygen at the gas inlet of the fixed-bed reactor 1 is not more than the limit oxygen concentration, and supply of the raw material gas is initiated next, and then the amount supplied of the raw material gas and the amount supplied of the molecular oxygen-containing gas are increased so that the concentration of the raw material gas at the gas inlet of the fixed-bed reactor 1 exceeds the upper explosion limit.

When the amounts supplied of the raw material gas and the molecular oxygen-containing gas are increased, the amount supplied of the mixed gas may be made constant by decreasing the amount supplied of water vapor. Thus, the time the gas resides in the pipings and the fixed-bed reactor 1 can be kept constant, and a change in pressure in the fixed-bed reactor 1 can be suppressed.

The pressure in the fixed-bed reactor 1 (specifically, the pressure at the gas inlet of the fixed-bed reactor 1) that is the pressure in the step (A) may preferably be not less than 0.1 MPaG and not more than 0.4 MPaG, more preferably not less than 0.15 MPaG and not more than 0.35 MPaG, further preferably not less than 0.2 MPaG and not more than 0.3 MPaG.

When the pressure in the step (A) is controlled to fall within the aforementioned range, the reaction efficiency in the oxidative dehydrogenation reaction improves.

When the pressure in the step (A) is excessively low, the reaction efficiency in the oxidative dehydrogenation reaction tends to decrease. On the other hand, when the pressure in the step (A) is excessively high, the yield in the oxidative dehydrogenation reaction tends to decrease.

In the oxidative dehydrogenation reaction, a gas hourly space velocity (GHSV) determined by the following expression (1) may preferably be not less than 500 h⁻¹ and not more than 5,000 h⁻¹, more preferably not less than 800 h⁻¹ and not more than 3,000 h⁻¹, further preferably not less than 1,000 h⁻¹ and not more than 2,500 h⁻¹.

When GHSV is controlled to fall within the aforementioned range, the reaction efficiency in the oxidative dehydrogenation reaction can improve further.

GHSV[h⁻¹]=gas flow rate at atmospheric pressure [Nm³/h]/catalyst layer volume [m³] Expression (1):

The “catalyst layer volume” in the aforementioned expression (1) represents a volume (apparent volume) of the whole catalyst layer containing pores.

Since an oxidative dehydrogenation reaction is an exothermic reaction, the temperature of a reaction system in the oxidative dehydrogenation reaction increases, and reaction by-products such as an unsaturated carbonyl compound with 3 to 4 carbon atoms may be produced. When the concentration of the unsaturated carbonyl compound with 3 to 4 carbon atoms in the produced gas is high, various adverse effects occur. Specifically, the unsaturated carbonyl compound dissolves in the absorption solvent and the like which are circulated and used in the step (C) described below. Thus, there is a tendency for an impurity to accumulate in the absorption solvent and the like, a deposit to be deposited on each member, or coking (deposition of solid carbon) to occur in the metal oxide catalyst.

As examples of a procedure for controlling the concentration of the unsaturated carbonyl compound to fall within a certain range in the oxidative dehydrogenation reaction, may be mentioned a method for adjusting the reaction temperature of the oxidative dehydrogenation reaction. When the reaction temperature is adjusted, the concentration of molecular oxygen at the gas outlet of the fixed-bed reactor 1 can be within a certain range.

Specifically, the reaction temperature may preferably be not lower than 300° C. and not higher than 400° C., more preferably not lower than 320° C. and lower than 380° C.

When the reaction temperature is controlled to fall within the aforementioned range, coking (deposition of solid carbon) can be suppressed in the metal oxide catalyst, and the concentration of the unsaturated carbonyl compound in the produced gas can be within a certain range. Furthermore, the concentration of molecular oxygen at the gas outlet of the fixed-bed reactor 1 can also be within a certain range.

On the other hand, when the reaction temperature is excessively low, the conversion rate of n-butene may decrease. When the reaction temperature is excessively high, the concentration of the unsaturated carbonyl compound increases, and there is a tendency for an impurity to accumulate in the absorption solvent and the like, or coking to occur in the metal oxide catalyst.

Herein, as specific preferable examples of the method for adjusting the reaction temperature, may be mentioned a procedure in which the fixed-bed reactor 1 is appropriately cooled by removing heat with a heating medium (specifically, dibenzyltoluene, a nitrite salt, or the like), to control the temperature of the catalyst layer to be constant.

Produced Gas:

The produced gas includes reaction by-products, the raw material gas unreacted, molecular oxygen unreacted, the gas for concentration adjustment and others in addition to 1,3-butadiene, which is a target product of the oxidative dehydrogenation reaction of the raw material gas and the molecular oxygen-containing gas. Examples of the reaction by-products include a carbonyl compound and a heterocyclic compound. Herein, the carbonyl compound includes ketones, aldehydes and organic acids.

As examples of the ketones, may be mentioned methyl vinyl ketone, acetophenone, benzophenone, anthraquinone and fluorenone.

As examples of the aldehydes, may be mentioned acetaldehyde, acrolein, methacrolein, crotonaldehyde and benzaldehyde.

As examples of the organic acids, may be mentioned maleic acid, fumaric acid, acrylic acid, phthalic acid, benzoic acid, crotonic acid, tetrahydrophthalic acid, isophthalic acid, terephthalic acid, methacrylic acid and phenol.

As examples of the heterocyclic compound, may be mentioned furan and cis-4-cyclohexene-1,2-dicarboxylic anhydride.

In the produced gas obtained in the step (A), the yield of the carbonyl compound may preferably be not more than 1.34 mol %, more preferably not more than 1.14 mol %.

In addition, in the produced gas obtained in the step (A), the yield of the heterocyclic compound may preferably be not more than 3.01 mol %, more preferably not more than 2.98 mol %.

When the yield of each component in the produced gas obtained in the step (A), that is, the produced gas discharged from a quench tower 2, falls within the aforementioned corresponding range, the efficiency of butadiene purification in the next and subsequent steps can improve and the side reaction of butadiene occurring during purification can be suppressed, thereby further reducing the energy consumption in producing butadiene.

Step (B):

In the step (B), the produced gas obtained in the step (A) is cooled. In this step (B), cooling of the produced gas from the step (A) is usually performed by the quench tower 2 and a heat exchanger for cooling 3, as illustrated in FIG. 1.

The step (B) will specifically be described. The produced gas from the step (A), that is, the produced gas discharged from the fixed-bed reactor 1 is fed to the quench tower 2 through the piping 101, cooled by the quench tower 2, then fed to the heat exchanger for cooling 3 through a piping 104, and further cooled by the heat exchanger for cooling 3. After the step (B) of cooling the produced gas by the quench tower 2 and the heat exchanger for cooling 3 as described above, the produced gas (hereinafter also referred to as “cooled produced gas”) is discharged from the heat exchanger for cooling 3 to a piping 105.

By undergoing this step (B), the produced gas from the step (A) is purified. Specifically, a part of the reaction by-products contained in the produced gas from the step (A) is removed.

Quench Tower:

The quench tower 2 is configured to bring the produced gas from the step (A) into countercurrent contact with a cooling medium to cool the produced gas to a temperature between about 30° C. and about 90° C. In the quench tower 2, a gas inlet for introducing the produced gas from the step (A) is provided at a lower portion, and a medium inlet for introducing the cooling medium is provided at an upper portion. The piping 101 having an end connected to the gas outlet of the fixed-bed reactor 1 is connected to the gas inlet, and a piping 102 is connected to the medium inlet. In the quench tower 2, a gas outlet for discharging the produced gas from the step (A) that has been cooled by the cooling medium is provided at a tower top, and a medium outlet for discharging the cooling medium which has been in contact (countercurrent contact) with the produced gas from the step (A) is provided at a tower bottom. The piping 104 is connected to the gas outlet, and a piping 103 is connected to the medium outlet.

In the example of this drawing, the cooling medium, which has been in contact (countercurrent contact) with the produced gas from the step (A) and discharged from the quench tower 2, is collected through the piping 103 and appropriately treated, thereby removing the reaction by-products (specifically, organic acids described below). Thus, the cooling medium is reused.

In the quench tower 2, for example, water or an aqueous alkali solution is used as a cooling medium.

The temperature of the cooling medium (temperature thereof at the medium inlet) is appropriately set depending on the intended cooling temperature, and may preferably be not lower than 10° C. and not higher than 90° C., more preferably not lower than 20° C. and not higher than 70° C., particularly preferably not lower than 20° C. and not higher than 40° C.

Furthermore, the temperature inside the quench tower 2 during operation may preferably be not lower than 10° C. and not higher than 100° C., more preferably not lower than 20° C. and not higher than 90° C.

In addition, it is preferable that the pressure in the quench tower 2 during operation (specifically, the pressure at the gas outlet of the quench tower 2), that is, the pressure in the step (B) is equal to or less than the pressure in the step (A).

Specifically, the difference between the pressure in the step (B) and the pressure in the step (A), that is, a value obtained by subtracting the value of the pressure in the step (B) from the value of the pressure in the step (A) may preferably be 0 MPaG or more and not more than 0.05 MPaG, more preferably not less than 0.01 MPaG and not more than 0.04 MPaG.

When the pressure difference between the step (A) and the step (B) is controlled to fall within the aforementioned range, condensation and dissolution into the cooling medium of the reaction by-products in the produced gas from the step (A) can be promoted in the quench tower 2. As a result, the concentration of the reaction by-products in the produced gas, discharged from the quench tower 2, can be further reduced.

Furthermore, the cooling medium, which has been in contact with the produced gas from the step (A) and discharged from the quench tower 2, contains organic acids. The organic acids are reaction by-products in the produced gas from the step (A) and have been condensed or dissolved in the cooling medium in the quench tower 2.

In the cooling medium having been in contact with the produced gas, that is, in the cooling medium discharged from the quench tower 2, it is preferable that the yield of the organic acids is not more than 2%. When the yield of the organic acids is excessively large, the load on the wastewater treatment of the cooling medium may increase.

Heat Exchanger for Cooling:

As the heat exchanger for cooling 3, a heat exchanger that is capable of cooling the produced gas, discharged from the quench tower 2, to room temperature (not lower than 10° C. and not higher than 30° C.) is appropriately used.

In the example of this drawing, the heat exchanger for cooling 3 has a gas inlet to which the piping 104, which has an end connected to the gas outlet of the quench tower 2, is connected and a gas outlet to which the piping 105 is connected.

It is preferable that the pressure in the heat exchanger for cooling 3 during operation (specifically, the pressure at the gas outlet of the heat exchanger for cooling 3) is equal to the pressure in the quench tower 2 during operation (the pressure at the gas outlet of the quench tower 2).

The concentration of molecular nitrogen in the cooled produced gas discharged from the heat exchanger for cooling 3 may preferably be not less than 60% by volume and not more than 94% by volume, more preferably not less than 70% by volume and not more than 85% by volume. The concentration of butadiene may preferably be not less than 2% by volume and not more than 15% by volume, more preferably not less than 3% by volume and not more than 10% by volume. The concentration of water (water vapor) may preferably be not less than 1% by volume and not more than 30% by volume, more preferably not less than 1% by volume and not more than 3% by volume. The concentration of the ketone and aldehydes may preferably be 0% by volume or more and not more than 0.3% by volume, more preferably not less than 0.05% by volume and not more than 0.25% by volume.

Step (C):

In the step (C), the produced gas cooled in the step (B) is separated (roughly separated) into molecular oxygen and inert gases, and the other gases containing 1,3-butadiene by selective absorption into the absorption solvent. Herein, the “other gases containing 1,3-butadiene” refer to a gas containing at least butadiene and n-butene (unreacted n-butene). Specifically, the other gases may contain the reaction by-products (specifically, ketones and aldehydes) in addition to butadiene and n-butene.

In this step (C), the separation of the cooled produced gas is performed by an absorption tower 4, as illustrated in FIG. 1. Herein, the absorption tower 4 is a tower in which a gas inlet for introducing the cooled produced gas is provided at a lower portion, a medium inlet for introducing the absorption solvent is provided at an upper portion, a liquid outlet for discharging the absorption solvent, which gases (specifically, the other gases containing 1,3-butadiene) are absorbed in, (hereinafter also referred to as “gas-absorbing liquid”) is provided at a tower bottom, and a gas outlet for discharging a gas that is not absorbed by the absorption solvent (specifically, molecular oxygen and inert gases) is provided at a tower top. The piping 105, which has an end connected to the gas outlet of the heat exchanger for cooling 3, is connected to the gas inlet. Furthermore, a piping 106 is connected to the medium inlet, a piping 113 is connected to the liquid outlet, and a piping 107 is connected to the gas outlet.

The step (C) will specifically be described. The cooled produced gas from the step (B), that is, the cooled produced gas discharged from the heat exchanger for cooling 3 is fed to the absorption tower 4 through the piping 105, and in synchronization with this feeding, the absorption solvent is supplied to the absorption tower 4 through the piping 106. Thus, the absorption solvent is brought into countercurrent contact with the cooled produced gas, and so the other gases containing 1,3-butadiene in the cooled produced gas are selectively absorbed by the absorption solvent. As a result, the other gases containing 1,3-butadiene, and the molecular oxygen and the inert gases are roughly separated. While the absorption solvent (the gas-absorbing liquid), which has absorbed the other gases containing 1,3-butadiene, is discharged to the piping 113, the molecular oxygen and the inert gases which have not been absorbed by the absorption solvent are discharged to the piping 107.

The temperature inside the absorption tower 4 during operation is not particularly limited. In general, molecular oxygen and inert gases are hardly absorbed by the absorption solvent as the temperature inside the absorption tower 4 increases. On the other hand, the absorption efficiency of hydrocarbons such as butadiene (the other gases containing 1,3-butadiene) into the absorption solvent increases as the temperature inside the absorption tower 4 decreases. Thus, the temperature inside the absorption tower 4 may preferably be not lower than 0° C. and not higher than 60° C., more preferably not lower than 10° C. and not higher than 50° C., in consideration of the productivity of butadiene.

In addition, it is preferable that the pressure in the absorption tower 4 during operation (specifically, the pressure at the gas outlet of the absorption tower 4), that is, the pressure in the step (C) is equal to or less than the pressure in the step (B).

Specifically, the difference between the pressure in the step (C) and the pressure in the step (B), that is, a value obtained by subtracting the value of the pressure in the step (C) from the value of the pressure in the step (B) may preferably be 0 MPaG or more and not more than 0.05 MPaG, more preferably not less than 0.01 MPaG and not more than 0.04 MPaG.

When the pressure difference between the step (B) and the step (C) is controlled to fall within the aforementioned range, absorption of butadiene (the other gases containing 1,3-butadiene) into the absorption solvent in the absorption tower 4 can be promoted. As a result, the amount of the absorption solvent used can be reduced and energy consumption can be reduced.

Absorption Solvent:

As the absorption solvent, those capable of selectively absorbing the other gases containing 1,3-butadiene are used.

Specifically, examples of the absorption solvent include those containing an organic solvent as a main component. As used herein, “containing an organic solvent as a main component” indicates that the content ratio of the organic solvent in the absorption solvent is not less than 50% by mass.

Examples of the organic solvent constituting the absorption solvent include an aromatic compound such as toluene, xylene and benzene, an amide compound such as dimethylformamide and N-methyl-2-pyrrolidone, a sulfur compound such as dimethyl sulfoxide and sulfolane, a nitrile compound such as acetonitrile and butyronitrile, and a ketone compound such as cyclohexanone and acetophenone.

The amount used (amount supplied) of the absorption solvent is not particularly limited, and may preferably be not less than 10 times by mass and not more than 100 times by mass, more preferably not less than 17 times by mass and not more than 40 times by mass, relative to the flow rate (mass flow rate) of the sum of butadiene and n-butene in the cooled produced gas.

When the amount used of the absorption solvent is controlled to fall within the aforementioned range, the absorption efficiency of the other gases containing 1,3-butadiene can improve.

On the other hand, when the amount used of the absorption solvent is excessively large, the energy consumption, which is used in purification for the absorption solvent to be circulated and used, tends to increase. In addition, when the amount used of the absorption solvent is excessively small, the absorption efficiency of the other gases containing 1,3-butadiene tends to decrease.

The temperature (temperature at the solvent inlet) of the absorption solvent may preferably be not lower than 0° C. and not higher than 60° C., more preferably not lower than 0° C. and not higher than 40° C.

When the temperature of the absorption solvent is controlled to fall within the aforementioned range, the absorption efficiency of the other gases containing 1,3-butadiene can further improve.

Circulation Step:

In the circulation step, the molecular oxygen and the inert gases obtained in the step (C) are appropriately treated, as necessary, and are fed as a reflux gas to the step (A). In this circulation step, the molecular oxygen and the inert gases from the step (C) are treated by a solvent-collecting tower 5 and a compressor 6.

This circulation step will specifically be described. The molecular oxygen and the inert gases from the step (C), that is, the molecular oxygen and the inert gases discharged from the absorption tower 4 are fed to the solvent-collecting tower 5 through the piping 107, subjected to a solvent removal treatment, and then fed to the compressor 6 through a piping 110. As necessary, a pressure adjustment treatment is performed. The molecular oxygen and the inert gases from the step (C) that have been subjected to the solvent removal treatment and the pressure adjustment treatment as described above are discharged from the compressor 6 to the piping 112 toward the reaction tower 1.

In the example of this drawing, while the molecular oxygen and the inert gases discharged from the solvent-collecting tower 5 pass through the piping 110, apart of the molecular oxygen and the inert gases is discarded through a piping 111 communicating with the piping 110. When the piping 111 for discarding a part of the molecular oxygen and the inert gases discharged from the solvent-collecting tower 5 is thus provided, the amount of the reflux gas to be supplied to the step (A) can be adjusted.

Solvent-Collecting Tower:

The solvent-collecting tower 5 is configured to wash the molecular oxygen and the inert gases from the step (C) with water or a solvent to perform the solvent removal treatment of the molecular oxygen and the inert gases. In the solvent-collecting tower 5, a gas inlet for introducing the molecular oxygen and the inert gases from the step (C) is provided at a central portion, and a water or solvent inlet for introducing water or a solvent is provided at an upper portion. The piping 107 having an end connected to the gas outlet of the absorption tower 4 is connected to the gas inlet, and a piping 108 is connected to the water or solvent inlet. In the solvent-collecting tower 5, a gas outlet for discharging the molecular oxygen and the inert gases washed with water or the solvent is provided at a tower top, and a water or solvent outlet for discharging the water or solvent used in washing the molecular oxygen and the inert gases from the step (C) is provided at a tower bottom. The piping 110 is connected to the gas outlet, and a piping 109 is connected to the water or solvent outlet.

In this solvent-collecting tower 5, the absorption solvent contained in the molecular oxygen and the inert gases from the step (C) is removed, and the thus removed absorption solvent is discharged to the piping 109 together with the water or solvent used for washing, so as to be collected through this piping 109. Furthermore, the molecular oxygen and the inert gases from the step (C), which have been subjected to the solvent removal treatment, are discharged to the piping 110.

In addition, the temperature inside the solvent-collecting tower 5 during operation is not particularly limited, and may preferably be not lower than 0° C. and not higher than 80° C., more preferably not lower than 10° C. and not higher than 60° C.

Compressor:

As the compressor 6, a compressor that is capable of increasing the pressure of the molecular oxygen and the inert gases from the solvent-collecting tower 5, as necessary, and adjusting the pressure to a pressure required in the step (A) is appropriately used.

In the example of this drawing, the compressor 6 has a gas inlet to which the piping 110, which has an end connected to the gas outlet of the solvent-collecting tower 5, is connected and a gas outlet to which the piping 112 is connected.

When the pressure in the step (C) is lower than the pressure in the step (A), pressurization by a pressure difference between the step (C) and the step (A) is performed by this compressor 6 according to the pressure difference.

When the pressurization is performed by this compressor 6, the pressure increase is usually small. Therefore, the electric energy consumption of the compressor is kept small.

In the molecular oxygen and the inert gases discharged from the compressor 6, that is, in the reflux gas, the concentration of molecular nitrogen may preferably be not less than 87% by volume and not more than 97% by volume, more preferably not less than 90% by volume and not more than 95% by volume. Furthermore, the concentration of the molecular oxygen may preferably be not less than 1% by volume and not more than 6% by volume, more preferably not less than 2% by volume or more and not more than 5% by volume.

Step (D):

In the step (D), the 1,3-butadiene liquid is obtained through steps (D1) and (D2) in this order from the gas-absorbing liquid obtained in the step (C). Also in the step (D1), a reusable absorption solvent is obtained through the steps (D1) and (D2), and a step (E) in this order. Herein, the liquid containing 1,3-butadiene obtained in the step (D) contains at least 1,3-butadiene and n-butane.

That is, in the step (D) including the steps (D1), (D2), and (E), the reusable absorption solvent is first obtained in the step (D1). Then, the 1,3-butadiene liquid is obtained in the step (D2), and the reusable absorption solvent is further obtained in the step (E).

Step (D1):

The absorption solvent is separated from the gas-absorbing liquid obtained in the step (C), and so, in the step (D1), the absorption solvent (hereinafter also referred to as “separated absorption solvent (D1)”) and a gas-absorbing liquid, which absorption components including the other gases containing 1,3-butadiene is concentrated in (hereinafter also referred to as “concentrated gas-absorbing liquid”), are obtained. That is, the gas-absorbing liquid from the step (C) is separated by distillation into the separated absorption solvent (D1) and the concentrated gas-absorbing liquid.

In this step (D1), the separation of the gas-absorbing liquid is performed by a desolvation tower 7, a condenser 8, and a reboiler 9, as illustrated in FIG. 1.

The step (D1) will specifically be described. The gas-absorbing liquid from the step (C), that is, the gas-absorbing liquid discharged from the absorption tower 4 is fed to the desolvation tower 7 through the piping 113 and separated by distillation. By the separation by distillation in this desolvation tower 7, the gas-absorbing liquid and the absorption solvent (hereinafter also referred to as “absorption solvent (D1)”) are obtained. A roughly separated concentrated gas discharged from the desolvation tower 7 is fed to the condenser 8 through a piping 115 and cooled, and the concentrated gas-absorbing liquid is discharged from the condenser 8 to a piping 119. On the other hand, the absorption solvent (D1) discharged from the desolvation tower 7 is fed to the reboiler 9 through a piping 114 and discharged from the reboiler 9 through a piping 118.

Desolvation Tower:

The desolvation tower 7 is configured so that the gas-absorbing liquid from the step (C) is separated by distillation. In the desolvation tower 7, a liquid inlet for introducing the gas-absorbing liquid from the step (C) is provided at a central portion. A gas outlet for discharging the roughly separated concentrated gas is provided at a tower top, and a liquid outlet for discharging the absorption solvent (D1) is provided at a tower bottom. The piping 113, which has an end connected to the liquid outlet of the absorption tower 4, is connected to the liquid inlet, the piping 115 is connected to the liquid outlet at the tower top, and the piping 114 is connected to the liquid outlet at the tower bottom.

In this desolvation tower 7, the gas absorbing liquid is separated by distillation into the roughly separated concentrated gas and the absorption solvent (D1). Then, the roughly separated concentrated gas is discharged to the piping 115, and the absorption solvent (D1) is discharged to the piping 114.

The pressure inside the desolvation tower 7 is not particularly limited, and the pressure may preferably be not less than 0.03 MPaG and not more than 1.0 MPaG, more preferably not less than 0.2 MPaG and not more than 0.6 MPaG.

The temperature of the desolvation tower 7 during operation at the tower bottom may preferably be not lower than 80° C. and not higher than 190° C., more preferably not lower than 100° C. and not higher than 180° C.

Condenser:

As the condenser 8, a condenser that is capable of further distilling the roughly separated concentrated gas-absorbing liquid from the desolvation tower 7 to concentrate the absorption component is appropriately used.

In the example of this drawing, the condenser 8 has a liquid inlet to which the piping 115, which has an end connected to the outlet at the top of the desolvation tower 7, is connected, and a liquid outlet in which the piping 119 and a circulation outlet are provided. A piping 117, which is the circulation outlet, has an end connected to the circulation outlet of the condenser 8 and another end connected to a circulation inlet provided at an upper portion of the desolvation tower 7. The piping 117 is for use in feeding the gas-absorbing liquid toward the desolvation tower 7.

Reboiler:

As the reboiler 9, a reboiler that is capable of heating the absorption solvent (D1) from the desolvation tower 7 is appropriately used.

The absorption solvent (D1) discharged from this reboiler 9 to the piping 118 is supplied again to the absorption tower 4 through a piping 133 and the piping 106 as it is without further purification.

In the example of this drawing, the reboiler 9 has a liquid inlet to which a part of the piping 114, which has an end connected to the liquid outlet of the desolvation tower 7, is connected, and a circulation outlet to which a piping 116 is connected. This piping 116 has an end connected to the circulation outlet of the reboiler 9 and another end connected to a circulation inlet provided at a lower portion of the desolvation tower 7.

The absorption solvent (D1) discharged from the reboiler 9 is substantially free of reaction by-products (specifically, free of ketones and aldehydes). Specifically, in the separated absorption solvent (D1), the concentration of the ketones and aldehydes is 0% by mass or more and not more than 1% by mass, and may preferably be 0% by mass or more and not more than 0.05% by mass.

When the concentration of the ketones and aldehydes in the separated absorption solvent (D1) falls within the aforementioned range, the separated absorption solvent (D1) can be used in the step (C) as it is without further purification.

In this step (D1), it is preferable that the amount of the gas-absorbing liquid to be subjected to the step (D1) is larger than the amount of the concentrated gas-absorbing liquid to be subjected to the step (D2).

Specifically, it is preferable that the ratio of the amount of the concentrated gas-absorbing liquid to be subjected to the step (D2) to the amount of the gas-absorbing liquid to be subjected to the step (D1) is 0.01 to 0.1.

Step (D2):

In the step (D2), the concentrated gas-absorbing liquid obtained in the step (D1) is separated by distillation into a 1,3-butadiene liquid containing 1,3-butadiene and a reaction by-product-containing solvent containing reaction by-products (specifically, the ketones and aldehydes).

In this step (D2), the concentrated gas-absorbing liquid is separated by a desolvation tower 10, a condenser 11, and a reboiler 12, as shown in FIG. 1.

The step (D2) will specifically be described. The concentrated gas-absorbing liquid from the step (D1), that is, the concentrated gas-absorbing liquid discharged from the condenser 8 is fed to the desolvation tower 10 through the piping 119 and separated by distillation. By the separation by distillation in this desolvation tower 10, an absorption solvent containing 1,3-butadiene and an absorption solvent containing reaction by-products are obtained. The absorption solvent containing 1,3-butadiene discharged from the desolvation tower 10 is fed to the condenser 11 through a piping 121 and cooled. Then, the 1,3-butadiene liquid is discharged from the condenser 11 to a piping 125. Herein, the 1,3-butadiene liquid may contain n-butene together with 1,3-butadiene. On the other hand, the absorption solvent containing reaction by-products, which is discharged from the desolvation tower 10, is fed to the reboiler 12 through a piping 122.

Desolvation Tower:

The desolvation tower 10 is configured so that the concentrated gas-absorbing liquid from the step (D1) is separated by distillation. In the desolvation tower 10, a liquid inlet for introducing the concentrated gas-absorbing liquid from the step (D1) is provided at a central portion. A liquid outlet for discharging a gas containing 1,3-butadiene is provided at a tower top, and a liquid outlet for discharging the absorption solvent containing the reaction by-products is provided at a tower bottom. The piping 119, which has an end connected to the liquid outlet of the condenser 8, is connected to the liquid inlet, the piping 121 is connected to the gas outlet at the tower top, and the piping 122 is connected to the liquid outlet at the tower bottom.

In this desolvation tower 10, the gas containing 1,3-butadiene and the absorption solvent containing reaction by-products, which have been separated from the concentrated gas absorbing liquid, are discharged to the piping 121 and the piping 122, respectively.

The pressure inside the desolvation tower 10 is not particularly limited, and the pressure may preferably be not less than 0.03 MPaG and not more than 1.0 MPaG, more preferably not less than 0.2 MPaG and not more than 0.6 MPaG.

The temperature of the desolvation tower 10 during operation at the tower bottom may preferably be not lower than 80° C. and not higher than 190° C., more preferably not lower than 100° C. and not higher than 180° C.

Condenser:

As the condenser 11, a condenser that is capable of cooling the gas containing 1,3-butadiene from the desolvation tower 10 is appropriately used.

In the example of this drawing, the condenser 11 has a liquid inlet to which the piping 121, which has an end connected to the outlet at the tower top of the desolvation tower 10, is connected, and a liquid outlet to which the piping 125 is connected. The condenser 11 has a circulation outlet. To the circulation outlet, a piping 123 is connected. This piping 123 has an end connected to the circulation outlet of the condenser 11 and another end connected to a circulation inlet provided at an upper portion of the desolvation tower 10. The piping 123 is for use in feeding the 1,3-butadiene liquid toward the desolvation tower 10.

Reboiler:

As the reboiler 12, a reboiler that is capable of heating the absorption solvent containing the reaction by-products from the desolvation tower 10 is appropriately used.

In the example of this drawing, the reboiler 12 has a liquid inlet to which a part of the piping 122, which has an end connected to the liquid outlet at the tower bottom of the desolvation tower 10, is connected. A piping 124 has an end connected to a circulation outlet of the reboiler 12 and another end connected to a circulation inlet provided at a lower portion of the desolvation tower 10. The piping 124 is for use in feeding the absorption solvent containing the reaction by-products toward the desolvation tower 10 from the reboiler 12.

It is preferable that the amount of the concentrated gas-absorbing liquid to be subjected to the step (D2) is smaller than the amount of the gas-absorbing liquid to be subjected to the step (D1), as described above.

Step (E):

In the step (E), a reaction by-product-containing liquid obtained in the step (D) is purified. In this step (E), the purification of the reaction by-product-containing liquid is performed by a solvent-collecting tower 13, a condenser 14, and a reboiler 15, as illustrated in FIG. 1.

The step (E) will specifically be described. The reaction by-product-containing liquid from the step (D) is fed to the solvent-collecting tower 13 through a piping 126 and separated by distillation. By the separation by distillation in this solvent-collecting tower 13, an absorption solvent contained in a trace amount in the reaction by-product-containing liquid is separated from the reaction by-product-containing liquid, to obtain the absorption solvent (hereinafter, also referred to as “absorption solvent (E)”) and the reaction by-product-containing liquid in which the reaction by-products are further concentrated. The absorption solvent (E) discharged from the solvent-collecting tower 13 is fed to the reboiler 15 through a piping 128, heated, and discharged to a piping 130. On the other hand, a concentrated reaction by-product-containing gas discharged from the solvent-collecting tower 13 is fed to a heat exchanger for solvent collection 14 through a piping 127 and cooled, and the reaction by-product liquid is discharged from the condenser 14 to a piping 129.

Solvent-Collecting Tower:

The solvent-collecting tower 13 is configured so that the reaction by-product-containing liquid from the step (D) is separated by distillation. In the solvent-collecting tower 13, an inlet for introducing the reaction by-product-containing liquid from the step (D) is provided at a central portion. A liquid outlet for discharging the concentrated reaction by-product-containing liquid is provided at a tower top, and a liquid outlet for discharging the absorption solvent (E) is provided at a tower bottom. The piping 126 having an end connected to the liquid outlet of the heat exchanger for concentration 12 is connected to the liquid inlet, the piping 127 is connected to the liquid outlet at the tower top, and the piping 128 is connected to the liquid outlet at the tower bottom.

In this solvent-collecting tower 13, the reaction by-product-containing liquid is separated by distillation into the concentrated reaction by-product-containing gas and the roughly separated absorption solvent (E). Then, the concentrated reaction by-product-containing gas is discharged to the piping 127, and the roughly separated absorption solvent (E) is discharged to the piping 128.

The pressure inside the solvent-collecting tower 13 is not particularly limited, and the pressure may preferably be not less than 0.03 MPaG and not more than 1.0 MPaG, more preferably not less than 0.2 MPaG and not more than 0.6 MPaG.

The temperature of the solvent-collecting tower 13 during operation at the tower bottom may preferably not lower than 80° C. and not higher than 190° C., more preferably not lower than 100° C. and not higher than 180° C.

Condenser:

As the condenser 14, a condenser that is capable of cooling the absorption solvent contained in a trace amount in the concentrated reaction by-product-containing liquid from the solvent-collecting tower 13 is appropriately used.

From such a condenser 14, the reaction by-products are discharged to the piping 129. The reaction by-product liquid discharged to this piping 129 is discarded.

In the example of this drawing, the condenser 14 has a liquid inlet to which the piping 127, which has an end connected to the liquid outlet of the solvent-collecting tower 13, is connected, and a reaction by-product liquid outlet to which the piping 129 is connected. The condenser 14 has a circulation outlet. To the circulation outlet, a piping 131 is connected. This piping 131 has an end connected to the circulation outlet of the condenser 14 and another end connected to a circulation inlet provided at an upper portion of the solvent-collecting tower 13. The piping 131 is for use in feeding the concentrated reaction by-product-containing liquid toward the solvent-collecting tower 13.

Reboiler:

As the reboiler 15, a reboiler that is capable of heating the absorption solvent (E) from the solvent-collecting tower 13 is appropriately used.

The absorption solvent (E) discharged from this reboiler 15 to the piping 130 is supplied again to the absorption tower 4 through the pipings 133 and 106 as it is.

In the example of this drawing, the reboiler 15 has a liquid inlet to which a part of the piping 128, which has an end connected to the liquid outlet of the solvent-collecting tower 13, is connected. The piping 130 is connected to a part of the piping 128 and communicates with the piping 106 via the piping 133. The reboiler 15 has a circulation outlet. To the circulation outlet, a piping 132 is connected. This piping 132 has an end connected to the circulation outlet of the reboiler 15 and another end connected to a circulation inlet provided at a lower portion of the solvent-collecting tower 13. The piping 132 is for use in feeding the absorption solvent (E) toward the solvent-collecting tower 13.

The absorption solvent (E) discharged from the reboiler 15 is returned to the step (C) together with the absorption solvent (D1) discharged from the reboiler 9. That is, the separated absorption solvent (E) discharged from the reboiler 15 to the piping 130 and the absorption solvent (D1) discharged from the reboiler 9 to the piping 118 are mixed in the piping 113, and the mixed absorption solvents are supplied again to the absorption tower 4 through the piping 106.

In the absorption solvent which is supplied again through the piping 133 and the piping 106 in this manner, it is preferable that the concentration of the ketones and aldehydes is 0% by mass or more and not more than 1% by mass.

According to the method for producing 1,3-butadiene of the present invention, since the molar ratio of water vapor to n-butene in the gases supplied to the fixed-bed reactor 1 is not more than 1.2, generation of reaction by-products such as carbonyl compounds and organic acids can be suppressed.

### EXAMPLES

Hereinafter, specific examples of the present invention will be described, but the present invention is not limited to these examples.

In the following Examples, gas composition analysis was performed by gas chromatography under conditions shown in Table 1 below. Regarding water vapor, calculation was performed by adding the moisture content obtained by water-cooling trapping during gas sampling.

TABLE 1
SUMMARY OF GAS COMPOSITION ANALYSIS

| GAS TYPE | 1,3-BUTADIENE, n-BUTENE | N₂, O₂, COx, H₂O |
|---|---|---|
| TYPE | GC-14B (MANUFACTURED BY | GC-14B (MANUFACTURED BY |
| | SHIMADZU CORPORATION) | SHIMADZU CORPORATION) |
| DETECTOR | FID | TCD |
| COLUMN | TC-BOND Alumina/Na₂SO₄ | WG-100 |
| | 0.53 mm I.D. × 30 m df = 10 μm | 6.35 mm I.D. × 1.8 m |
| | (MANUFACTURED BY | (MANUFACTURED BY |
| | GL SCIENCES INC.) | GL SCIENCES INC.) |
| CARRIER GAS | N₂ 40 ml/min | He 50 ml/min |
| TEMPERATURE | | |
| INJECTION | 200° C. | 60° C. |
| DETECTOR | 250° C. | 80° C. |
| COLUMN | 60° C. 5 min→135° C.(5° | 50° C. |
| | C./min)→185° C.(15° C./min) | |

Analysis of a carbonyl compound, analysis of a heterocyclic compound, and analysis of an organic acid were performed by liquid chromatography under conditions shown in Table 2 below.

TABLE 2

| | |
|---|---|
| TYPE | LC-2000Plus (MANUFACTURED BY JASCO |
| | CORPORATION) |
| DETECTOR | UV (210 nm, 230 nm) |
| COLUMN | TSKgel ODS-100V 5 μm |
| | 4.6 mmID × 15 cm |
| | (MANUFACTURED BY TOSOH CORPORATION) |
| ELUENT | ACETONITRILE/PHOSPHORIC ACID LIQUID |
| | 0.8 ml/min |
| COLUMN OVEN | 40° C. |

The conversion rate of n-butene, the yield of butadiene, and analysis of aldehydes were performed by gas chromatography under the conditions of Table 1.

### Example 1

In accordance with the flow diagram of FIG. 1, 1,3-butadiene was produced from a raw material gas containing n-butene through the following steps (A), (B), (C), (D1), (D2), and (E), and circulation step.

Step (A):

To the fixed-bed reactor 1 (inner diameter: 21.2 mm, outer diameter: 25.4 mm) filled with a metal oxide catalyst so that the length of a catalyst layer was 4,000 mm, a mixed gas with a volume ratio (n-butene/O₂/N₂/H₂O) of 1/1.5/17.5/0 (the molar ratio of water vapor to n-butene was 0) was supplied at a gas hourly space velocity (specifically, SV calculated using a flow rate in a standard state) of 2,000 h⁻¹. The raw material gas and a molecular oxygen-containing gas were subjected to an oxidative dehydrogenation reaction under a condition of a reaction temperature of 320 to 330° C., to obtain a produced gas containing 1,3-butadiene. The pressure in this step (A), that was, the pressure at the gas inlet of the fixed-bed reactor 1 was 0.1 MPaG.

In this step (A), as the metal oxide catalyst, a metal oxide catalyst in which an oxide represented by a composition formula: Mo₁₂Bi₅Fe_{0.5}Ni₂Co₃K_{0.1}Cs_{0.1}Sb_{0.2} was carried on spherical silica at a proportion of 20% of the whole catalyst volume was used.

As the mixed gas, the raw material gas and a reflux gas (molecular oxygen and inert gases) were mixed, and as necessary, air as the molecular oxygen-containing gas, molecular nitrogen as the inert gases, and water (water vapor) were further mixed, to adjust a composition.

Step (B):

The produced gas discharged from the fixed-bed reactor 1 was brought into countercurrent contact with water as a cooling medium to be quenched in the quench tower 2. After being cooled to 76° C., the produced gas was cooled to 30° C. in the heat exchanger 3.

Step (C):

The produced gas discharged from the heat exchanger for cooling 3 (cooled produced gas) was supplied from the gas inlet at the lower portion of the absorption tower 4 (outer diameter: 152.4 mm, height: 7,800 mm, material: SUS304) inside of which a regular packing was disposed, and an absorption solvent containing toluene in an amount of not less than 95% by mass was supplied at 10° C. from the solvent inlet at the upper portion of the absorption tower 4. The amount of the absorption solvent supplied was 33 times by mass the flow rate (mass flow rate) of the sum of butadiene and n-butene in the cooled produced gas. The pressure in this step (C), that was, the pressure at the gas outlet of the absorption tower 4 was 0.1 MPaG.

Circulation Step:

The gas discharged from the absorption tower 4 was washed with water or a solvent in the solvent-collecting tower 5, to remove a small amount of absorption solvent contained in the gas. The gas in which the absorption solvent was thus removed was discharged from the solvent-collecting tower 5, a part of the gas was discarded, and most of the rest was fed to the compressor 6. In the compressor 6, the gas from the solvent-collecting tower 5 was pressurized by a pressure adjustment treatment. The absorption solvent was thus removed, and the pressurized gas was discharged from the compressor 6 and returned to the reaction tower 1.

Step (D): Step (D1):

The liquid discharged from the absorption tower 4 was separated by distillation in the desolvation tower 7, and the separated gas discharged from the outlet at the tower top of the desolvation tower 7 was cooled by the condenser 8, to obtain a concentrated liquid (hereinafter also referred to as “concentrated separated liquid (D1)”). On the other hand, the absorption solvent (hereinafter also referred to as “circulating absorption solvent (D1)”) was obtained at the liquid outlet at the tower bottom of the desolvation tower 7.

Step (D): Step (D2):

The concentrated separated liquid (D1) discharged from the condenser 8 was separated by distillation in a separation tower 10, and the separated gas discharged from the outlet at the tower top of the desolvation tower 10 was cooled by the condenser 11, to obtain a 1,3-butadiene liquid containing 1,3-butadiene. This 1,3-butadiene liquid was collected as a production target. On the other hand, a concentrated liquid containing reaction by-products (hereinafter also referred to as “concentrated separated liquid (D2)”) was obtained from the liquid outlet at the tower bottom of the desolvation tower 10.

Step (D): Step (E):

The concentrated separated liquid (D2) discharged from the liquid outlet at the tower bottom of the desolvation tower 10 was separated and purified in the solvent-collecting tower 13, and the absorption solvent (hereinafter also referred to as “circulating absorption solvent (E)”) was obtained from the liquid outlet at the tower bottom of the solvent-collecting tower 13. On the other hand, the separated gas discharged from the outlet at the tower top of the solvent-collecting tower 13 was cooled by the condenser 14, to obtain a reaction by-product liquid containing the reaction by-products. The reaction by-product liquid was discarded.

The circulating absorption solvent (E) obtained in this step (E) was supplied to the absorption tower 4 through the pipings 133 and 106 together with the circulating absorption solvent (D1) obtained in the step (D1).

The conversion rate of n-butene and the yield of 1,3-butadiene in the step (A), the yield of a carbonyl compound and the yield of a heterocyclic compound in the produced gas obtained in the step (A), and the yield of organic acids in a drainage in the step (B) in the above description are shown in Table 3 below.

### Example 2

The same procedures as those of Example 1 were performed to produce 1,3-butadiene, except that the mix gas supplied to the fixed-bed reactor 1 was changed to a gas with a volume ratio (n-butene/O₂/N₂/H₂O) of 1/1.5/16.9/0.6 (the molar ratio of water vapor to n-butene was 0.6).

The conversion rate of n-butene and the yield of 1,3-butadiene in the step (A), the yield of a carbonyl compound and the yield of a heterocyclic compound in the produced gas obtained in the step (A), and the yield of organic acids in a drainage in the step (B) are shown in Table 3 below.

### Example 3

The same procedures as those of Example 1 were performed to produce 1,3-butadiene, except that the mix gas supplied to the fixed-bed reactor 1 was changed to a gas with a volume ratio (n-butene/O₂/N₂/H₂O) of 1/1.5/16.3/1.2 (the molar ratio of water vapor to n-butene was 1.2).

The conversion rate of n-butene and the yield of 1,3-butadiene in the step (A), the yield of a carbonyl compound and the yield of a heterocyclic compound in the produced gas obtained in the step (A), and the yield of organic acids in a drainage in the step (B) are shown in Table 3 below.

### Comparative Example 1

The same procedures as those of Example 1 were performed to produce 1,3-butadiene, except that the mix gas supplied to the fixed-bed reactor 1 was changed to a gas with a volume ratio (n-butene/O₂/N₂/H₂O) of 1/1.5/15.1/2.4 (the molar ratio of water vapor to n-butene was 2.4).

The conversion rate of n-butene and the yield of 1,3-butadiene in the step (A), the yield of a carbonyl compound and the yield of a heterocyclic compound in the produced gas obtained in the step (A), and the yield of organic acids in a drainage in the step (B) are shown in Table 3 below.

TABLE 3

| | | | | COMPARATIVE |
|---|---|---|---|---|
| | EXAMPLE 1 | EXAMPLE 2 | EXAMPLE 3 | EXAMPLE 1 |
| GAS HOURLY SPACE VELOCITY(GHSV) [h⁻¹] | 2000 | 2000 | 2000 | 2000 |
| COMPOSITION OF MIXED GAS (MOLAR RATIO) | 1/1.5/17.5/0 | 1/1.5/16.9/0.6 | 1/1.5/16.3/1.2 | 1/1.5/15.1/2.4 |
| n-BUTENE/O₂/N₂/H₂O | | | | |
| MOLAR RATIO OF WATER VAPOR TO n-BUTENE | 0 | 0.6 | 1.2 | 2.4 |
| IN MIXED GAS [H2O/n-BUTENE] | | | | |
| CONVERSION RATE OF n-BUTENE | 84.10 | 83.99 | 83.63 | 82.80 |
| IN STEP A (MOL %) | | | | |
| YIELD OF BUTADIENE IN STEP A (MOL %) | 71.17 | 70.46 | 69.81 | 68.46 |
| YIELD OF CARBONYL COMPOUND IN | 0.98 | 1.14 | 1.34 | 1.69 |
| PRODUCED GAS OBTAINED IN STEP A (MOL %) | | | | |
| YIELD OF HETEROCYCLIC COMPOUND IN | 2.89 | 2.98 | 3.01 | 3.43 |
| PRODUCED GAS OBTAINED IN STEP A (MOL %) | | | | |
| YIELD OF ORGANIC ACIDS IN DRAINAGE | 1.38 | 1.72 | 2.00 | 2.37 |
| IN DRAINAGE IN STEP B (MOL %) | | | | |

From results shown in Table 3, it was confirmed that according to a method for producing 1,3-butadiene of Examples 1 to 3, the generation of reaction by-products was suppressed, and 1,3-butadiene was obtained at a high yield.

### REFERENCE SIGNS LIST

1 fixed-bed reactor
2 quench tower
3 heat exchanger for cooling
4 absorption tower
5 solvent-collecting tower
6 compressor
7 desolvation tower
8 condenser
9 reboiler
10 desolvation tower
11 condenser
12 reboiler
13 solvent-collecting tower
14 condenser
15 reboiler
21 desolvation tower
100 to 132, 140 piping

1: A method for producing 1,3-butadiene, the method comprising:
(A) performing an oxidative dehydrogenation reaction, with oxygen in the presence of a metal oxide catalyst, of a raw material gas, which comprises n-butene, to obtain a produced gas comprising 1,3-butadiene;
(B) cooling the produced gas obtained in (A); and
(C) separating the produced gas cooled in (B) into molecular oxygen and inert gases, and other gases comprising 1,3-butadiene, by selective absorption into an absorption solvent, wherein:
in (A), the raw material gas and a molecular oxygen-containing gas, as a minimum, are supplied to a fixed-bed reactor, wherein an inside of the fixed-bed reactor includes the metal oxide catalyst, which is a composite oxide catalyst comprising molybdenum and bismuth;
a molar ratio of molecular oxygen to n-butene (molecular oxygen/n-butene) in the gases supplied to the fixed-bed reactor is 1.0 to 2.0; and
a molar ratio of water vapor (water) to n-butene (water/n-butene) in the gases supplied to the fixed-bed reactor is not more than 1.2.

2: The method for producing 1,3-butadiene according to claim 1, wherein in the produced gas obtained in (A), a yield of a carbonyl compound is not more than 1.34 mol %, and a yield of a heterocyclic compound is not more than 3.01 mol %.

3: The method for producing 1,3-butadiene according to claim 1, wherein in (B), the produced gas comes into contact with a cooling medium so that the produced gas is cooled, and an yield of organic acids in the cooling medium that has been in contact with the produced gas is not more than 2 mol %.

4: The method for producing 1,3-butadiene according to claim 1, wherein in (A), the molar ratio of water vapor to n-butene in the gases supplied to the fixed-bed reactor is not more than 0.6.
